(19) United States
(12) Patent Application Publication
(10) Pub. No.: US 2026/0151126 A1
(43) Pub. Date: Jun. 4, 2026

(54) SUTURE RETAINING SYSTEM AND METHOD

(71) Applicant: Stitchlock, Inc., Webster, TX (US)

(72) Inventors: Clayton L. Moliver, Houston, TX (US); Gerald Baty, The Woodlands, TX (US); Mark M. Gittleman, Houston, TX (US)

(21) Appl. No.: 19/387,027

(22) Filed: Nov. 12, 2025

Related U.S. Application Data
(60) Provisional application No. 63/726,371, filed on Nov. 29, 2024.

(51) Int. Cl. A61B 17/04

(52) U.S. Cl. CPC A61B 17/0469; A61B 17/0482; A61B 2017/0409; A61B 2017/0414; A61B 2017/0456; A61B 2017/0464

(57) ABSTRACT

The present disclosure describes a system and method capable of facilitating wound closure during a surgical procedure without the need for suture tie offs or knots. In one embodiment, the present invention provides a retaining body capable of receiving and securing two ends of a suture in a desired location. In one embodiment, the retaining member has a first channel running an interior length of the retaining body for receiving the first portion of the suture and a second channel positioned on the bottom of the retaining body for receiving the second portion of the suture. The channel may include one or more teeth capable of engaging and securing the suture when it is inserted into the channel. The teeth may be lockable in order to secure the suture within the channel at a desired location.

### CROSS REFERENCE TO RELATED APPLICATIONS

This patent application claims priority upon and incorporates by reference herein, a provisional patent application entitled “SUTURE LOCKING DEVICES,” filed on Nov. 29, 2024, Ser. No. 63/726,371.

### BACKGROUND

Surgery is a complicated endeavor. Surgeons must possess not only extensive knowledge regarding the patient and the procedure in question, but also the physical dexterity to accomplish the objective of the surgery. Further, the surgeon must be able to close the surgical area, i.e., the wound, in a manner that promotes healing, avoids infection and is aesthetically pleasing. Wounds may be closed using one or more staples, sutures and/or adhesives as needed, depending on the type and depth of the wound, the type of tissue, the degree of tension required, etc.

Sutures are widely utilized in the surgical field due to their flexibility and ability to stay in place despite movement of the surrounding tissue. Sutures may be attached to a needle or threaded separately there-through. The needle is used by the surgeon to guide the suture in and out of the tissue so that the tissue is held together in the desired position by the suture(s). The sutures may be applied continuously, i.e., as a single suture weaving in and out of the tissue, or as a series of interrupted sutures, i.e., a separate suture for each section of the wound. Regardless of what type of suture is used, the suturing process takes time and requires outstanding fine motor skills on the part of the surgeon.

When suturing a wound, one of the challenges comes when the suture has to be tied off or knotted. Suture loops associated with knot tying may lead to ischemia from strangulated tissue and increased risk of dehiscence or rupture at the surgical wound. For example, if the suture is tied off too tightly, the suture may weaken, break and/or the knotted suture may push through the skin after a subcutaneous closure.

If the suture knot is too large, there may be increased risk of scarring/irritation of the surrounding tissue as well as a greater chance of infection due to surface contact between the suture knot and the surrounding tissue. These risks often increase where multiple surgeons are involved in the procedure, as each surgeon has his or her own preferences for suture type, tightness, knot characteristics, etc., often resulting in uneven suture(s) and/or suture tie offs.

As such, there remains a need for a suture retaining system and method capable of facilitating wound closure during a surgical procedure without the need for suture tie offs or knots. Eliminating the need for suture tie offs or knots will reduce the time required for surgical personnel to complete the surgical procedure while simultaneously reducing the risk of infection and potential complications relating thereto.

### SUMMARY

Accordingly, the present disclosure describes a system and method capable of facilitating wound closure during a surgical procedure without the need for suture tie offs or knots.

In one embodiment, the present invention provides a retaining body capable of receiving and securing two ends of a suture in a desired location in order to facilitate the closure of a wound without the need for suture tie offs or knots. In one embodiment, the retaining member has a first aperture for receiving a first portion of a suture and a second aperture for receiving a second portion of a suture.

The first aperture may include a channel running an interior length of the retaining body for receiving the first portion of the suture. The channel may include one or more teeth capable of engaging and securing the suture when it is inserted into the channel. The teeth may be lockable in order to secure the suture within the channel at a desired location.

The second aperture may include a second channel positioned on the bottom of the retaining body in a substantially perpendicular configuration with respect to the first channel. One or more locking members may be provided to lock the sutures into place after the surgical wound is closed.

This summary is provided to introduce a selection of concepts in a simplified form that are further described herein. This summary is not intended to identify key or essential features of the claimed subject matter, nor is it intended to be used as an aid in determining the scope of the claimed subject matter.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings; it being understood that the drawings contained herein are not necessarily drawn to scale and that the accompanying drawings provide illustrative implementations and are not meant to limit the scope of various technologies described herein; wherein:

FIG. 1 is a left side, top, front perspective view of the retaining body of one embodiment shown in an unlocked state.

FIG. 2A is a cross sectional view of FIG. 1 taken along line A-A.

FIG. 2B is a cross sectional view of FIG. 3 taken along line B-B.

FIG. 3 is a left side, top, front perspective view of the retaining body of one embodiment shown in a locked state.

FIG. 4A is a side view of the retaining body of one embodiment illustrating the locking members in an open position.

FIG. 4B is a side view of the retaining body of one embodiment illustrating the locking members in a partially closed position.

FIG. 4C is a side view of the retaining body of one embodiment illustrating the locking members in a closed position.

FIG. 5 is a left side, bottom, front perspective view of the retaining body of one embodiment shown in an unlocked state.

FIG. 6 is a left side, top, rear perspective view of the retaining body of one embodiment shown in an unlocked state.

FIGS. 7-10 illustrate the use of the retaining body in conjunction with a surgical method of one embodiment of the invention.

### DETAILED DESCRIPTION

In the following description, numerous details are set forth to provide an understanding of the present invention. However, it will be understood by those skilled in the art that the inventions described herein may be practiced without these details and that numerous variations or modifications from the described embodiments may be possible.

The present disclosure describes embodiments of a suture retaining system and a method of facilitating wound closure during a surgical procedure. In one embodiment, the suture retaining system of the present invention is capable of engaging both ends of a suture during a surgical procedure and locking them into the desired position without the need for suture knots or tie offs.

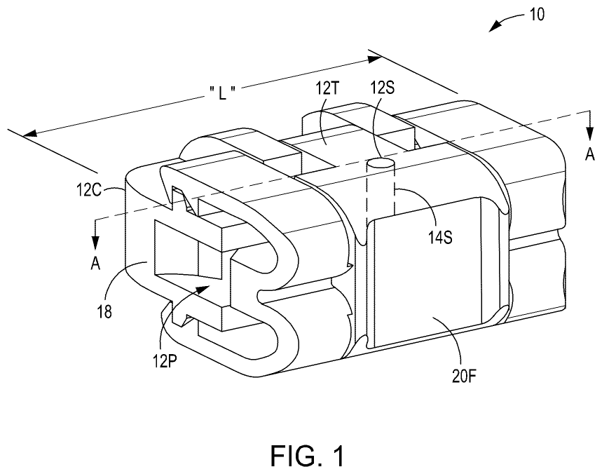

FIG. 1 illustrates one embodiment of the present invention including a retaining body (10) having one or more apertures (12P) for receiving a first portion (22A) of a suture (22). In the example of FIG. 1, the retaining body (10) is shown in an “unlocked” state without any sutures attached thereto. This feature of the present invention allows medical personnel to customize the state of the device, i.e., where and what type of suture(s) are to be attached, prior to, or during, the surgical procedure in question. However, it should be understood that the retaining body may also be provided to medical personnel with one or more suture(s) pre-attached to the retaining body, as described further below.

The retaining body and/or the sutures may be made from any number of materials, including bio-absorbable materials that break down in the body or non-absorbable materials that may be permanent and/or non-degradable, such as polypropylene. Non-absorbable sutures may be utilized where greater tensile strength is required and/or for wounds requiring prolonged wound healing and/or prolonged physical support. Absorbable sutures may be utilized where suture removal might affect the repair or where long-term physical support is unnecessary after complete wound healing.

In one embodiment, the aperture (12P) may include a channel (14) located within the interior of the retaining body. In one embodiment, the channel (14) may run the length (L) of the retaining body (10) and may be sized to receive a first portion (22A) of a suture (22). In one embodiment, the channel may also be capable of receiving a suture that has been attached to a needle (26). Such needle(s) may be of any suitable size/shape. For example, a straight needle or a curved needle in the shape of a hook may be utilized, as desired.

One of the many advantages provided by the present invention is the ability to receive a variety of sutures/needles of varying sizes/shapes in order to accommodate the preferences of surgical personnel. For example, the retaining body may be capable of receiving braided absorbable sutures (for example vicryl sutures), monofilament absorbable sutures, like monocryl or PDS, and/or barbed absorbable sutures. Further, the retaining body and/or the sutures may contain (or be coated with) one or more drugs to promote healing and/or prevent infection. One or more drug eluding devices (not shown) may also be utilized.

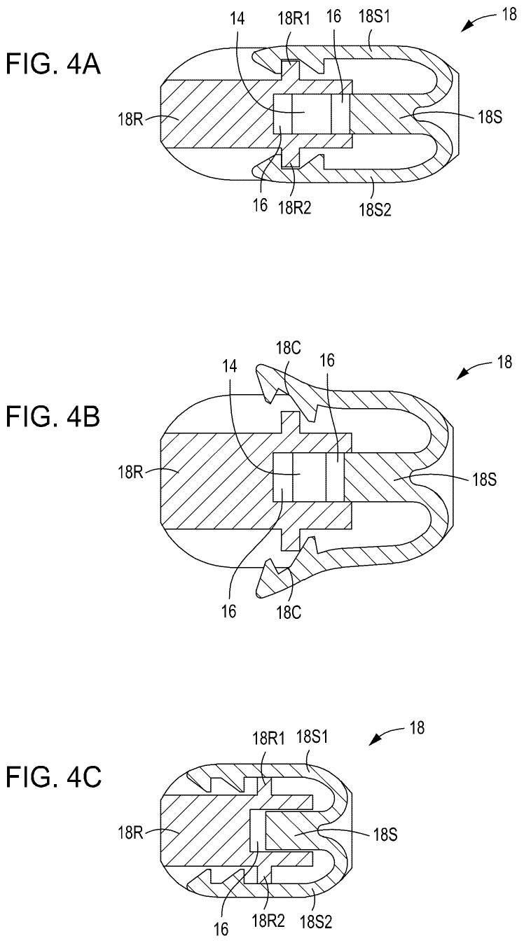

In one embodiment, the channel (14) may include one or more teeth (16) capable of engaging the suture (22) when it is inserted into the channel (14). As noted above, FIG. 1 illustrates the retaining body in an unlocked state. In an unlocked state, the teeth are held apart so that the suture (and the attached needle if desired) may be passed through the channel (14). When the retaining body is switched from an unlocked state to a locked state (see FIG. 3) the teeth engage the suture and retain it at the desired location within the channel (14).

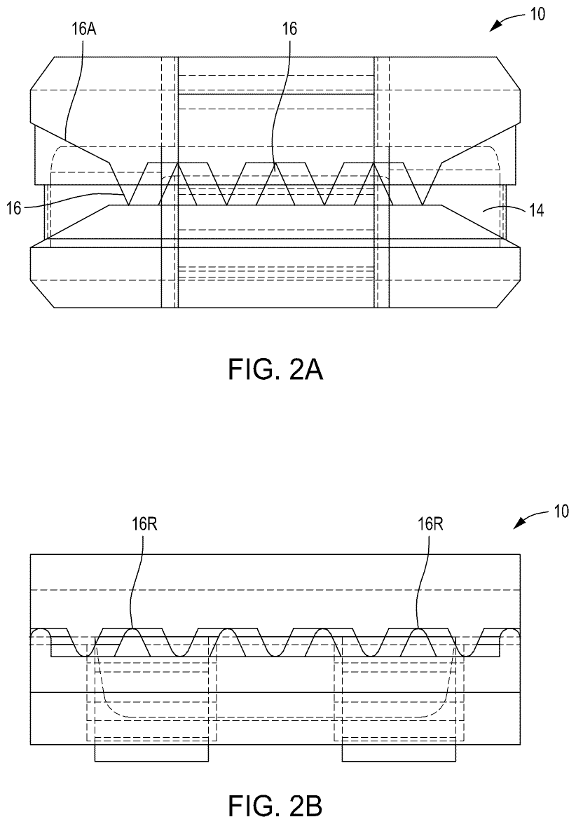

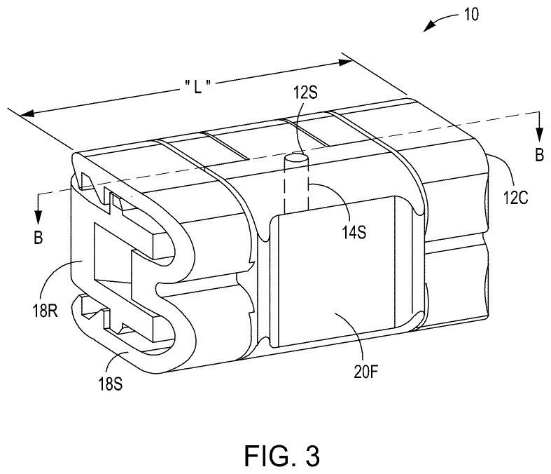

FIG. 2A illustrates an example configuration of the teeth (16) in one embodiment within the channel (14) of the retaining body (10), taken along line “A-A” of FIG. 1, with the retaining body in an unlocked state. FIG. 2B illustrates an example configuration of teeth in one embodiment, taken along line “B-B” of FIG. 3 where the retaining body is in a locked state, i.e., where the suture has been secured within the channel (14) at a desired position.

In one embodiment, one or more of the teeth (16) may have at least one rounded edge (16R) and/or have an angled (16A) configuration to provide clearance for the suture (22) and/or curvature of the needle (26). The use of rounded teeth, in this embodiment, allows the suture to be secured inside the channel at a desired location without being severed by the teeth when the retaining body is switched from an unlocked state to a locked state. Further, this feature of the invention allows the suture to be securely held in place without being “sawed” by the teeth (due to patient movement, etc.) during post-surgery activity.

In one embodiment, the present invention is equipped with one or more locking members (18) capable of securing the suture inside the channel at a desired location. In one embodiment, the locking members may be used to move the retaining body from an unlocked state (shown in FIG. 1) to a locked state (shown in FIG. 3).

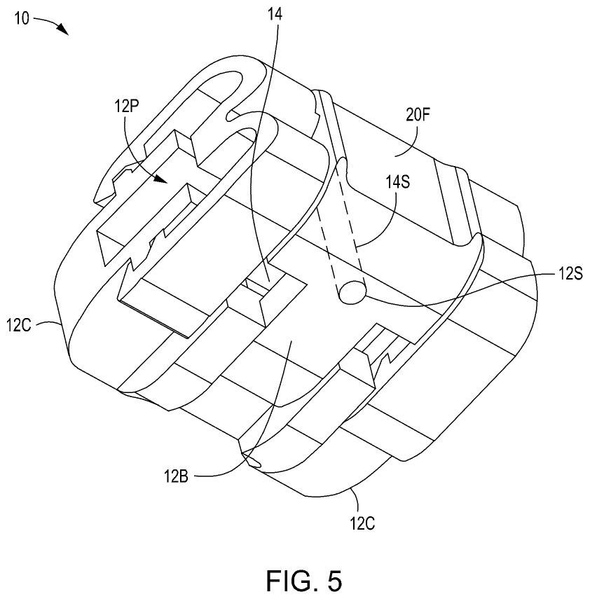

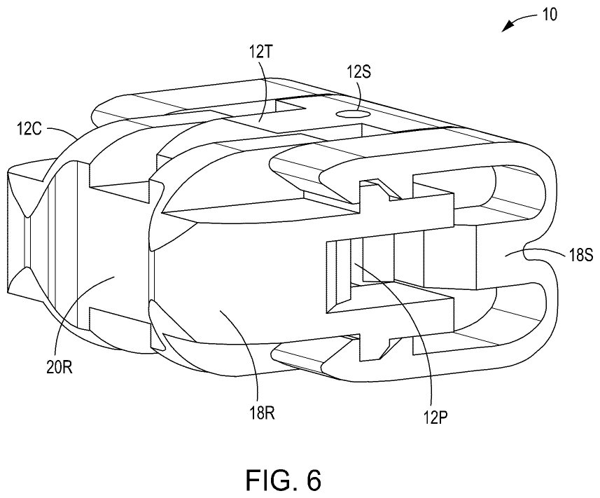

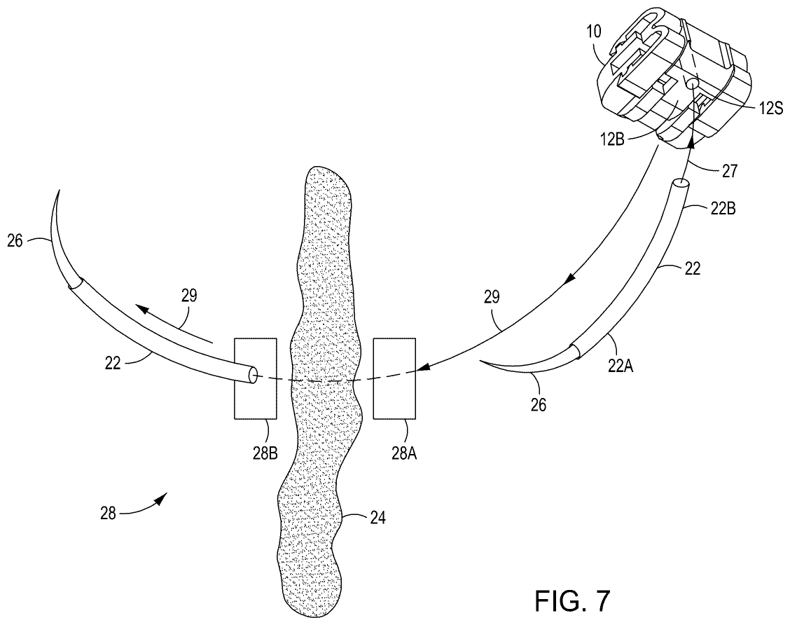

FIG. 4A illustrates an example locking member (18) of one embodiment of the present invention. The locking member of FIG. 4A shows the locking member (18) in an open position, which would be associated with the retaining body (10) being in a unlocked state.

In FIG. 4A, the example locking member (18) has a sliding section (18S) and a receiving section (18R). In one embodiment, the sliding section has a pair of deformable latches (18S1 and 18S2) disposed longitudinally on the proximal end thereof which may be engaged with a pair of ledges (18R1 and 18R2) on the receiving section (18R). As noted above, in one embodiment, one or more teeth (16) may be disposed inside the channel (14). When the retaining body is in an unlocked state, the teeth are held apart such that the channel has a width that provides ample clearance for the needle and suture. This enables the surgeon to pass the needle through the channel and adjust the tension of the suture, as desired.

FIG. 4B illustrates an example locking member (18) of one embodiment of the present invention where the locking member is in a partially closed position. In one embodiment, each of the deformable latches (18S1 and 18S2) may have a slanted surface (18C) which acts as a ramp enabling the latches to slide up the respective ledges (18R1 and 18R2) in a partially closed position as the slidable section (18S) moves from an open position to a closed position. As the slidable section moves, the channel (14) narrows, bringing the teeth closer together, which prescribes how much compression occurs on the suture disposed inside the channel.

FIG. 4C illustrates an example locking member (18) of one embodiment of the present invention where the locking member is in a closed position. This is how the example locking member of FIG. 4A would appear when the retaining body is in a locked state.

In this example, the pair of deformable latches (18S1 and 18S2) have slid to a position distal to the respective pair of ledges (18R1 and 18R2) and the channel has narrowed such that the suture is compressed in multiple locations by the teeth so as to hold the suture in the desired location.

In one embodiment, the retaining body (10) may be equipped with one or more grasping surfaces to facilitate the transition of the retaining body from an unlocked state to a locked state. In one embodiment, grasping surfaces are provided on the front (20F) and rear (20R) of the retaining body. In one embodiment, the grasping surfaces may be shaped and sized to engage the tips of a needle driver (not shown). The size and shape of the grasping surfaces, in this embodiment, make it easier for the surgeon to move the retaining body (10) from an unlocked state to a locked state as the tips of his or her needle driver will easily fit into the grasping surfaces to activate the locking members (18).

It should be noted that the grasping surfaces may be sized and/or shaped to engage any number of surgical tools. For example, the grasping surfaces may be designed to engage one or more robotic end effectors (not shown). In this example, robotic end effectors may be any device(s) attached to the end of a robotic arm that allow the robot to interact with its environment, functioning as the “hands” of the robot. In one embodiment, one or more of the grasping surfaces (or other portions of the retaining body) may be equipped with one or more sensors (not shown) designed to interact with sensors located on one or more robotic end effectors.

In one embodiment, the retaining body may have a substantially rectangular configuration. The retaining body may also be equipped with one or more convex corners (12C). In one embodiment, one or more exterior surfaces of the retaining body may be composed of a smooth, low friction material. In one embodiment, the retaining body may be composed of smooth plastic having a dynamic coefficient of friction of less than or equal to 0.33.

The present invention may include a number of unique innovations intended to improve the experience of the surgeon as well as the patient both during, and after, a surgical procedure. For example, the grasping surfaces being shaped and sized to receive the tip of a needle driver makes it much more likely for the surgeon to engage the grasping surfaces on the first try . . . making it less likely that the surgeon will accidentally grasp the patient's skin, organs or other items in the surgical area.

Further, the convex corners (12C) and the use of smooth, low friction surfaces make it less likely that the retaining body will accidentally “get caught” on the patient's skin, organs or other items while in the surgical area. These advantages are even more pronounced for surgeries that occur inside a small area of the patient's body (where space is at a premium) such as endoscopic surgery, laparoscopic surgery, etc. Further, the smooth, low friction design of the retaining body lowers the risk of problems during recovery because the smooth surfaces of the retaining body (10) are less likely to accidentally “get caught” on items in the patient's environment, thus reducing the risk of suture tear, etc.

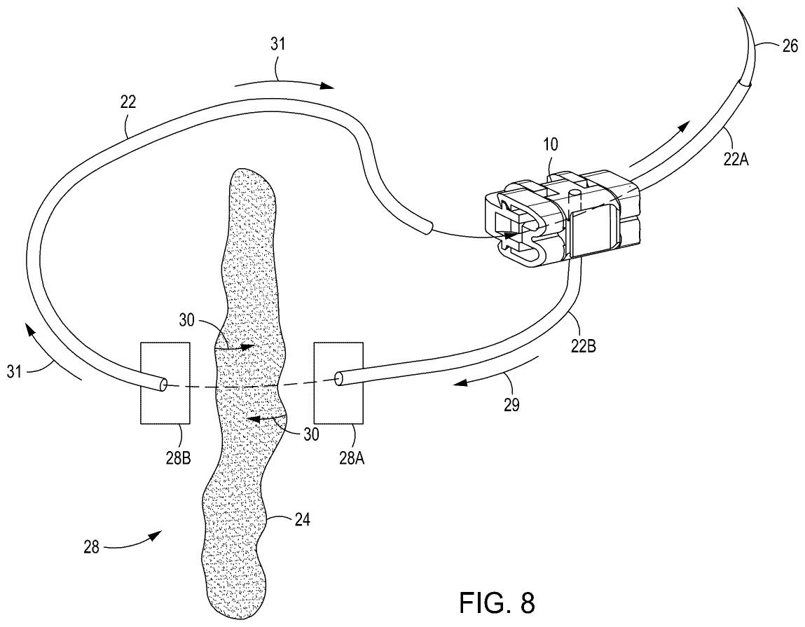

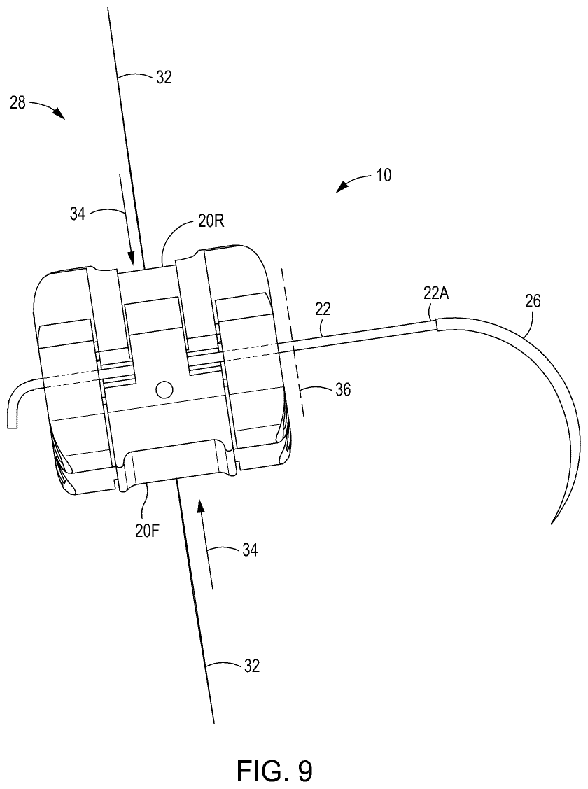

Referring to FIGS. 5-6, in one embodiment, the retaining body of the present invention may include one or more secondary apertures (12S) capable of receiving and securing a second portion (22B) of the suture (22). In one embodiment, the second portion of the suture may be pre-attached to the secondary aperture or insertable into the retaining body by the user prior to a surgical procedure.

In one embodiment, the second portion of the suture (22B) may be attached to a secondary aperture (12S) positioned on the top (12T) or bottom (12B) surface of the retaining body (10). This may be accomplished via any suitable method, including but not limited to, injection molding, over-molding, welding, ultrasonic welding, etc. In one embodiment, the secondary aperture (12S) may extend into the retaining body such that it forms a secondary channel (14S).

The secondary channel may extend into a portion of the retaining body (10) or span the entire height of the retaining body. In one embodiment, the secondary channel (14S) formed by the secondary aperture (12S) may be positioned in a substantially perpendicular configuration with respect to the channel (14) used to receive the first portion of the suture (22A). In another embodiment, the secondary aperture may be equipped with its own set of teeth (not shown) for engaging and retaining the suture.

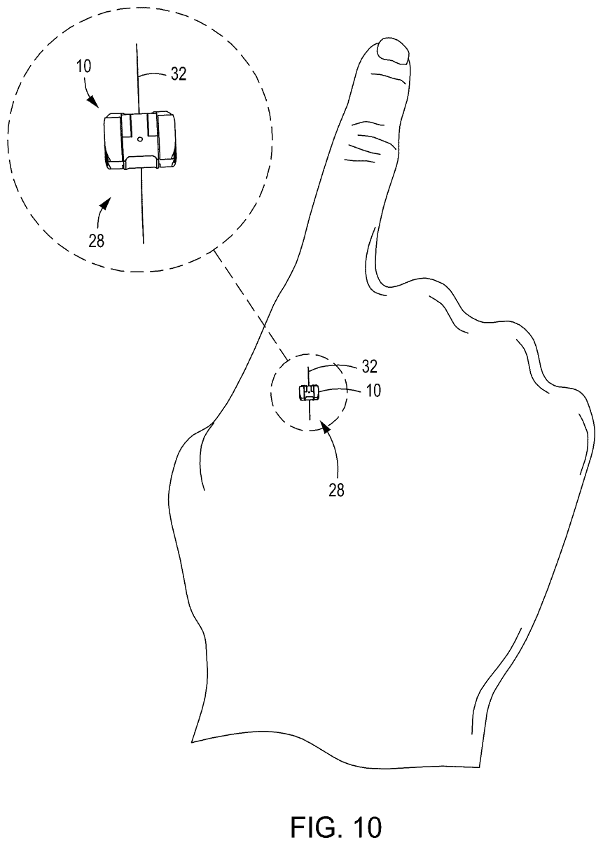

Referring to FIG. 7, the present disclosure further describes a method of facilitating wound closure during a surgical procedure without the use of suture knots or tie offs. In one embodiment, the method begins with providing the retaining body (10) described herein to the user. As described above, the retaining body (10) may be provided to the user in a stand alone configuration, i.e., no suture (22) attached, or with a portion of a suture (22B) pre-attached to the device.

In order to utilize the invention, a surgical area (28) having separated tissue (24) may be provided. The surgical area may include separated tissue relating to any type/field of surgery, including but not limited to minimally invasive surgeries (such as endoscopic surgery, laparoscopic surgery, robotic surgery, etc.) and/or open surgical procedures. Further, the surgery may be in any number of fields, such as veterinary medicine, field medicine and/or low/absent gravity procedures.

First, one end (22B) of a suture (22) may be attached to the retaining body (10), as illustrated by arrow (27). The suture may be attached at any suitable location of the retaining body. In one embodiment, one end of the suture may be attached to the secondary aperture (12S) of the retaining body (10). A needle (26) may be attached to the other end of the suture (22A) and the needle (pulling the suture and the attached retaining body behind it) may be inserted into the surgical area on the insertion side (28A) of the separated tissue and removed from the surgical area on the extraction side (28B) of the separated tissue (24), as illustrated by arrows (29). Referring to FIG. 8, once extracted, the needle (26) may be pulled away from the separated tissue (24) until the retaining body (10) is pulled into contact with the insertion side (28A) of the surgical area (28), as indicated by arrows (31).

The present invention includes a number of unique innovations intended to improve the experience of the surgeon as well as the patient both during, and after, a surgical procedure. For example, attaching the suture to the bottom (12B) of the retaining body (10) (in this example) in a substantially perpendicular configuration relative to the channel (14) predisposes the bottom surface (12B) of the retaining body (10) to lay flat against the surgical area when the retaining body is pulled into contact thereto. This feature of the present invention is highly advantageous as the surgeon does not have to adjust the position of the retaining body to make sure it is resting on the surgical area in the desired orientation. This advantage is even more pronounced for surgeries that occur inside a small area of the patient's body (where space is at a premium) such as endoscopic surgery, laparoscopic surgery, etc.

The needle (26) may then be inserted into and through the channel (14) of the retaining body (10). In one embodiment, the entire needle and the suture attached thereto may be inserted and drawn through the length (L) of the retaining body (10) through the channel (14). The needle may then be pulled away from the separated tissue (24). Doing so will cause the suture (22) to pull the insertion side (28A) and extraction side (28B) of the separated tissue (28) together, thus closing the wound, as illustrated by arrows (30) of FIG. 8.

Referring to FIG. 9, with the wound closed (32), the retaining body may be switched from an unlocked state to a locked state to lock the suture in the desired location. In one embodiment, this may be accomplished by pushing the grasping members (20F and 20R) together, illustrated by arrows (34). As noted above, in one embodiment, the grasping members may be sized and/or shaped to receive the tips of a needle driver and/or other tools (not shown).

In one embodiment, pushing the grasping members together activates the locking members (18) on one or both sides of the retaining body (10), which in turn, causes the teeth (16) inside the retaining body to come together inside the channel (14), thus holding the suture (22) at the desired location therein. Any suture remaining outside the retaining body may then be cut (illustrated by dotted line (36)) and removed from the surgical area. FIG. 10 illustrates an up-close view of the retaining body in a locked state, holding the wound closed, and a wide view of the surgical area showing the retaining body, in a locked state, on the hand of an example patient.

Although the invention has been described with reference to specific embodiments, this description is not meant to be construed in a limited sense. Various modifications of the disclosed embodiments, as well as alternative embodiments of the invention, will become apparent to persons skilled in the art upon reference to the description of the invention. It is, therefore, contemplated that the appended claims will cover such modifications that fall within the scope of the invention.

What is claimed is:

1. A suture retaining system comprising:
a retaining body having an aperture for receiving a needle attached to a suture;
wherein the aperture comprises an interior channel running a length of the retaining body such that the needle and the suture attached thereto may be passed through the length of the retaining body through the channel; wherein the channel comprises one or more securing members for releasably engaging the suture inside the channel; and
wherein the retaining body further comprises one or more locking members for securing the suture inside the channel at a desired position.

2. The system of claim 1, wherein the one or more securing members further comprise a plurality of teeth.

3. The system of claim 2, wherein one or more of the plurality of teeth are rounded.

4. The system of claim 1, wherein the retaining body further comprises a second aperture for receiving a second portion of the suture and wherein the second aperture is positioned on a bottom surface of the retaining body.

5. The system of claim 4, wherein the second aperture has a substantially perpendicular orientation relative to the bottom surface of the retaining body.

6. The system of claim 1, wherein the retaining body has a substantially rectangular configuration and wherein at least one corner of the retainer body has a convex configuration.

7. The system of claim 1, wherein at least a portion of an exterior surface of the retaining body comprises a low friction material having a dynamic coefficient of friction less than or equal to 0.33.

8. The system of claim 1, wherein the needle further comprises a hook.

9. The system of claim 1, wherein the retaining body further comprises two grasping surfaces for activating the locking members.

10. The system of claim 9, wherein the grasping surfaces are positioned on a front surface and a rear surface of the retaining body and each are sized to engage tips of a needle driver.

11. The system of claim 1, wherein the retaining body and the suture are absorbable by the human body.

12. A surgical method comprising the steps of:
providing a retaining body having an aperture for receiving a needle attached to a suture; wherein the aperture comprises an interior channel running a length of the retaining body such that the needle and the suture attached thereto may be passed through the length of the retaining body through the channel;
wherein the channel comprises one or more securing members for releasably engaging the suture inside the channel; and wherein the retaining body further comprises one or more locking members for securing the suture inside the channel at a desired position;
wherein the retaining body further comprises a second aperture for receiving a second portion of the suture;
attaching the second portion of the suture to the second aperture of the retaining body;
providing separated tissue;
inserting the needle into the separated tissue on a first side;
extracting the needle from a second side of the separated tissue;
pulling the needle away from the separated tissue until the retaining body is pulled into contact with the first side of the separated tissue;
inserting the needle into and through the aperture;
pulling the needle away from the separated tissue until the suture holds the first side and the second side of the separated tissue together;
activating one or more of the locking members to secure the suture inside the retaining body; and
cutting the suture.

13. The method of claim 12, wherein the surgical method is utilized during endoscopic surgery.

14. The method of claim 12, wherein the retaining body further comprises two grasping surfaces for activating the locking members; each of the grasping surfaces being sized to engage the tips of a needle driver.

15. The method of claim 12, wherein the one or more securing members further comprise a plurality of teeth.

16. The method of claim 15, wherein one or more of the plurality of teeth are rounded.

17. The system of claim 12, wherein the second aperture is positioned on a bottom surface of the retaining body.

18. The system of claim 17, wherein the second aperture has a substantially perpendicular orientation relative to the bottom surface of the retaining body.

19. The method of claim 13, wherein the needle further comprises a hook.

20. The method of claim 13, wherein the retaining body and the suture are absorbable by the human body.
